(19) United States
(12) Patent Application Publication
(10) Pub. No.: US 2007/0078509 A1
(43) Pub. Date: Apr. 5, 2007

(54) Valved balloon stent

(76) Inventor: Wael Mohamed Nabil Lotfy, Cairo (EG)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/572,737

(22) PCT Filed: Nov. 23, 2003
(86) PCT No.: PCT/EG03/00010

§ 371 Date: Mar. 21, 2006

(30) Foreign Application Priority Data
Sep. 24, 2003 (EG) 2003090946

(51) Int. Cl. A61F 2/06

(52) U.S. Cl. 623/1.24

(57) ABSTRACT

This invention is concerned with a valved inflatable balloon stent that is fixed through interventional cathterization to function as a valve e.g. cardiac valve.

This invention is concerned with a lined inflatable and dilatable valved balloon stent (the stent is dilatable and its lining is either inflatable or dilatable, the balloon is inflatable and deflatable) that will be introduced inside vessels to function as a valve.

So that the stent is introduced in its smaller size then dilated to take the size of the intended vessel; the balloon will then be inflated with an appropriate material e.g. carbon dioxide, normal saline, air.

Apart from the central balloon the proximal opening can be inflatable. In simpler terms, it's a cage like design mounted on the dilatable stent having a narrower opening on one side and the cage from the other side (the narrowing can be inflatable) and an inflatable ball enclosed. This ball functions as the valve.

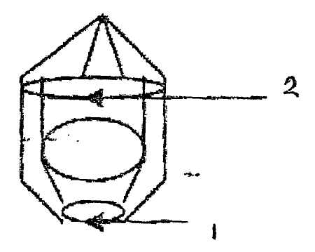

FIG. 1 demonstrates a sketch of the invention.

For this purpose the metallic dilatable stents in common use in cardiology practice can be modified to this new shape. The balloon moving inside the cage can be prepared from any inflatable and non reactive tissue e.g. similar to valvotomy balloons in common practice.

During placement of this valved balloon stent, the previous damaged non functioning valve can be crushed (putting the new valve in the place of the old one exactly).

This procedure will be done through per catheter intervention in the catheterization laboratory. It will allow emergency as well as permanent valve replacement when other options are worrisome.

I expect it thus to revolutionize the practice. Because the ability to perform per catheter inflatable valve replacement without mortality will definitely make surgical corrections of simple as well complicated cardiac lesions be not needed or at least deferrable to the time where they could be done with less mortality.

Previous State of the Art

A surgical procedure is undertaken with its inherent costs, risks and problems to replace the non functioning valve with another human, animal or metallic. Fixing an animal origin valve through catheterization.

Problems in the Previous State of the Art

High cost and associated risks of surgical operation for valve replacement. Non malleability in dealing with the valve after its placement. The high cost of the valve itself.

What is New About this Invention?

Achieve the same result of surgery through interventional catheterization. Malleability in the dealing with the valve during and after placement. Avoiding the risks and costs of the surgical or operation.

How Can it Be Used?

A selected company producing the common use intravascular stent will be chosen after agreement with the inventor to upgrade some of its stents with the new designs and linings I suggested.

1. The addition of inflatable and/or compressible and/or controllable lining to stents (medical or non medical) to function as a valve for the flow of fluids or gases through.
a. This includes any form of stents including but not limited to metallic, plastic, totally inflatable stents or otherwise of medical or non medical use.
b. This includes all shapes of stent designs including but not limited to ring, tubular, cylindrical, cone, pentagonal . . . etc.
c. This includes all shapes and materials of linings used for the same purpose including but not limited to Gortex, Teflon, PTFE.

2. The addition of fixed lining narrowing excluding animal native or treated valves to stents (medical or non medical) to function as a valve for the flow of fluids or gases through.
a. This includes any form of stents including but not limited to metallic, plastic, totally inflatable stents or otherwise of medical or non medical use.
b. This includes all shapes of stent designs including but not limited to ring, tubular, cylindrical cone, pentagonal . . . etc.
c. This includes all shapes and materials of linings used for the same purpose including but not limited to Gortex, Teflon, PTFE.

3-6. (canceled)

7. The ball for the valve mechanism is inflatable by CO2, air, flowable gelatinous material, metallic powder, radioopaque fluid or hardening agent.

8. The ball for the valve mechanism comprising a check valve for inflation or deflation.

9. The inflatable ball wherein the check valve for inflation is of a breakaway design to permit separation from the means for injecting.

10. The ball for the valve mechanism where the one way valve comprises a plug of an elastomer having a slit through which closes upon application of pressure within the tubing.

11. The ball for the valve mechanism where the ball is linked to the stent by a ribbon of biologically inert material to allow limited mobility of the ball and/or inflation or deflation of the ball alone or with the stent.

12. The ball for the valve mechanism where the ball is separate from the stent.

13. The ball for the valve mechanism that is modifiable and retrievable after implantation to allow further sizing as needed.

14. The lining of claim 1 and 2 that is fabricated solely or at least partly from a semipermeable membrane, and wherein the hollow wall has disposed hydrophilic material capable of absorbing a liquid to thereby increase the volume of said material. The final shape may be appropriate or modifiable by ballooning from the lumen or by inflation.

15. The lining of claim 14 that is fabricated from a semipermeable membrane, and wherein the hollow wall has disposed hydrophilic material that is a gel.

16. The use of ultrashort stents (whether fixed, balloonable or inflatable) i.e. rings to support the valve mechanism instead of usual stents.
